(19) United States
(12) Patent Application Publication
(10) Pub. No.: US 2015/0173825 A1
(43) Pub. Date: Jun. 25, 2015

(54) DEBRIDEMENT DEVICE HAVING A SPLIT SHAFT WITH BIOPOLAR ELECTRODES

(71) Applicant: Medtronic Xomed, Inc., Jacksonville, FL (US)

(72) Inventor: Eliot F. Bloom, Hopkinton, NH (US)

(73) Assignee: Medtronic Xomed, Inc., Jacksonville, FL (US)

(21) Appl. No.: 14/137,342

(22) Filed: Dec. 20, 2013

(51) Int. Cl. A61B 18/14

(52) U.S. Cl. CPC A61B 18/14; A61B 2018/1452

(57) ABSTRACT

A device includes an inner shaft rotatable within an outer shaft for cutting tissue. Additionally, the device can deliver energy including bipolar radiofrequency energy for sealing tissue which may be concurrent with delivery of fluid to a targeted tissue site. An inner shaft can be formed of two portions separated by an insulating layer. The portions define electrodes for delivery of energy.

### BACKGROUND

Concepts presented herein generally relate to devices, systems and methods for cutting and sealing tissue such as bone, cartilage, and soft tissue. These concepts can particularly suitable for sinus applications and nasopharyngeal/laryngeal procedures and may combine or provide radiofrequency energy delivery with a microdebrider device.

Devices, systems and methods according to the present disclosure may be suitable for a variety of procedures including ear, nose and throat (ENT) procedures, head and neck procedures, otology procedures, including otoneurologic procedures. The present disclosure may be suitable for a variety of other surgical procedures including mastoidectomies and mastoidotomies; nasopharyngeal and laryngeal procedures such as tonsillectomies, trachael procedures, adenoidectomies, laryngeal lesion removal, and polypectomies; for sinus procedures such as polypectomies, septoplasties, removals of septal spurs, anstrostomies, frontal sinus trephination and irrigation, frontal sinus opening, endoscopic DCR, correction of deviated septums and trans-sphenoidal procedures; rhinoplasty and removal of fatty tissue in the maxillary and mandibular regions of the face, as well as other procedures utilizing RF energy delivery.

Sinus surgery is challenging due to its location to sensitive organs such as the eyes and brain, the relatively small size of the anatomy of interest to the surgeon, and the complexity of the typical procedures. Examples of debriders with mechanical cutting components are described in U.S. Pat. Nos. 5,685,838; 5,957,881 and 6,293,957. These devices are particularly successful for powered tissue cutting and removal during sinus surgery, but do not include any mechanism for sealing tissue to reduce the amount of bleeding from the procedure. Sealing tissue is especially desirable during sinus surgery which tends to be a complex and precision oriented practice.

Electrosurgical technology was introduced in the 1920's. In the late 1960's, isolated generator technology was introduced. In the late 1980's, the effect of RF lesion generation was well known. See e.g., Cosman et al., Radiofrequency lesion generation and its effect on tissue impedance, Applied Neurophysiology (1988) 51: 230-242. Radiofrequency ablation is successfully used in the treatment of unresectable solid tumors in the liver, lung, breast, kidney, adrenal glands, bone, and brain tissue. See e.g., Thanos et al., Image-Guided Radiofrequency Ablation of a Pancreatic Tumor with a New Triple Spiral-Shaped Electrode, Cardiovasc. Intervent. Radiol. (2010) 33:215-218.

The use of RF energy to ablate tumors or other tissue is known. See e.g., McGahan J P, Brock J M, Tesluk H et al., Hepatic ablation with use of radio-frequency electrocautery in the animal model. J Vasc Intery Radiol 1992; 3:291-297. Products capable of aggressive ablation can sometimes leave undesirable charring on tissue or stick to the tissue during a surgical procedure. Medical devices that combine mechanical cutting and an electrical component for cutting, ablating or coagulating tissue are described, for example, in U.S. Pat. Nos. 4,651,734 and 5,364,395.

Commercial medical devices that include monopolar ablation systems include the Invatec MIRAS RC, MIRAS TX and MIRAS LC systems previously available from Invatec of Italy. These systems included a probe, a grounding pad on the patient and a generator that provides energy in the range of 450 to 500 kHz. Other examples of RF bipolar ablation components for medical devices are disclosed in U.S. Pat. Nos. 5,366,446 and 5,697,536.

Medical devices are also used to ablate heart tissue with RF energy. See, e.g., Siefert et al. Radiofrequency Maze Ablation for Atrial Fibrillation, Circulation 90(4): I-594. Some patents describing RF ablation of heart tissue include U.S. Pat. Nos. 5,897,553, 6,063,081 and 6,165,174. Devices for RF ablation of cardiac tissue are typically much less aggressive than RF used to cut tissue as in many procedures on cardiac tissue, a surgeon only seeks to kill tissue instead of cutting or removing the tissue. Cardiac ablation of this type seeks to preserve the structural integrity of the cardiac tissue, but destroy the tissue's ability to transfer aberrant electrical signals that can disrupt the normal function of the heart.

Transcollation® technology, for example, the sealing energy supplied by the Aquamantys® System (available from Medtronic Advanced Energy of Portsmouth, N.H.) is a patented technology which stops bleeding and reduces blood loss during and after surgery and is a combination of radiofrequency (RF) energy and saline that provides hemostatic sealing of soft tissue and bone and may lower transfusion rates and reduce the need for other blood management products during or after surgery. Transcollation® technology integrates RF energy and saline to deliver controlled thermal energy to tissue. Coupling of saline and RF energy allows a device temperature to stay in a range which produces a tissue effect without the associated charring found in other ablation methods.

Other ablation devices include both mechanical cutting as well as ablation energy. For example, the PK Diego® powered dissector is commercially available from Gyms ENT of Bartlett, Tenn. This device utilizes two mechanical cutting blade components that are moveable relative to each other, one of which acts as an electrode in a bipolar ablation system. The distal end portion of the device includes six layers to accomplish mechanical cutting and electrical coagulation. The dual use of one of the components as both a mechanical, oscillating cutting element and a portion of the bipolar system of the device is problematic for several reasons. First, the arrangement exposes the sharp mechanical cutting component to tissue just when hemostasis is sought. In addition, the electrode arrangement does not provide for optimal application of energy for hemostasis since the energy is applied essentially at a perimeter or outer edge of a cut tissue area rather than being applied to a central location of the cut tissue. The arrangement of the device also requires more layers than necessary in the construction of a device with both sharp cutters and RF ablation features. The overabundance of layers can make it difficult to design a small or optimally-sized distal end. Generally speaking, the larger the distal end, the more difficult it is for the surgeon to visualize the target tissue. The use of six layers at the distal end of the system also interferes with close intimate contact between the tissue and the electrodes. Some examples of cutting devices are described in U.S. Pat. Nos. 7,854,736 and 7,674,263.

The Medtronic Straightshot® M4 Microdebrider uses sharp cutters to cut tissue, and suction to withdraw tissue. While tissue debridement with the Medtronic microdebrider system is a simple and safe technique, some bleeding may occur. The Medtronic microdebrider does not include a feature dedicated to promoting hemostasis or bleeding management. Thus, nasal packing is often used.

### BRIEF DESCRIPTION OF THE DRAWINGS

FIG. 1 is a perspective view of a system;

FIG. 2 is a perspective view of an inner shaft and outer shaft of a device with the inner shaft in a first position;

FIG. 3 is a perspective view of the inner shaft and the outer shaft of the device with the inner shaft in a second position;

FIG. 4 is a perspective view of an inner shaft prior to separation;

FIG. 5 is a perspective view of the inner shaft after separation into two portions;

FIG. 6 is a perspective view of the inner shaft after an overmolding process;

FIG. 7 is a perspective view of the inner shaft with masking applied thereto;

FIG. 8 is a perspective view of the inner shaft after application of an insulating coating; and

FIG. 9 is a sectional view of the inner shaft along line 9-9 of FIG. 8.

### DETAILED DESCRIPTION

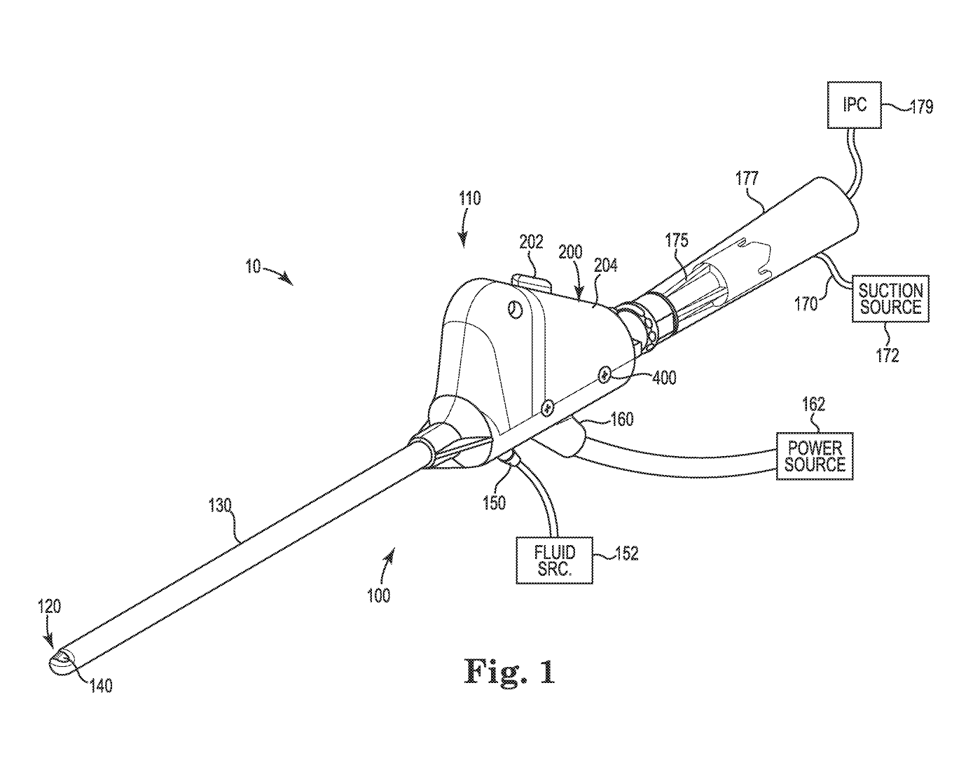

FIG. 1 illustrates a system 10 that includes a device 100 having a proximal end region indicated generally at 110 and a distal end region indicated generally at 120. The device includes an outer shaft 130 and an inner shaft 140 coaxially maintained within the outer shaft 130. A portion of the inner shaft 140 is shown in FIG. 1 at distal end region 120. Proximal end region 110 includes a button activation cell 200 comprising a button 202 maintained by a housing 204, the proximal end region further comprising a hub 175 coupled to inner shaft 140. The hub is configured to couple to a handle or handpiece 177 which can be manipulated by a user (e.g., a surgeon). The handpiece 177, in turn may be coupled to an integrated power console or IPC 179 for driving the device 100 and specifically for controlling rotation of inner shaft 140. The IPC 179 may also include a fluid source (not shown) and may provide fluid delivery to device 100.

Proximal end region 110 also includes a fluid source connector 150, a power source connector 160 and a suction source connector 170 for connection to a fluid source 152, a power source, 162 and/or a suction source 172 of system 10. One fluid useful with the device 100 is saline, however, other fluids are contemplated. Power source 162 may be a generator and optionally may be designed for use with bipolar energy or a bipolar energy supply. For example, the Transcollation® sealing energy supplied by the Aquamantys® System (available from Medtronic Advanced Energy of Portsmouth, N.H.) may be used. Both the fluid source 152 and suction source 172 are optional components of system 10. However, use of fluid in conjunction with energy delivery aids in providing optimal tissue effect as will be further explained. In use, a fluid (e.g., saline) may be emitted from an opening at the distal end region of the device 100. Tissue fragments and fluids can be removed from a surgical site through an opening (not shown in FIG. 1) in the distal end region via the suction source 172, as will be further explained below.

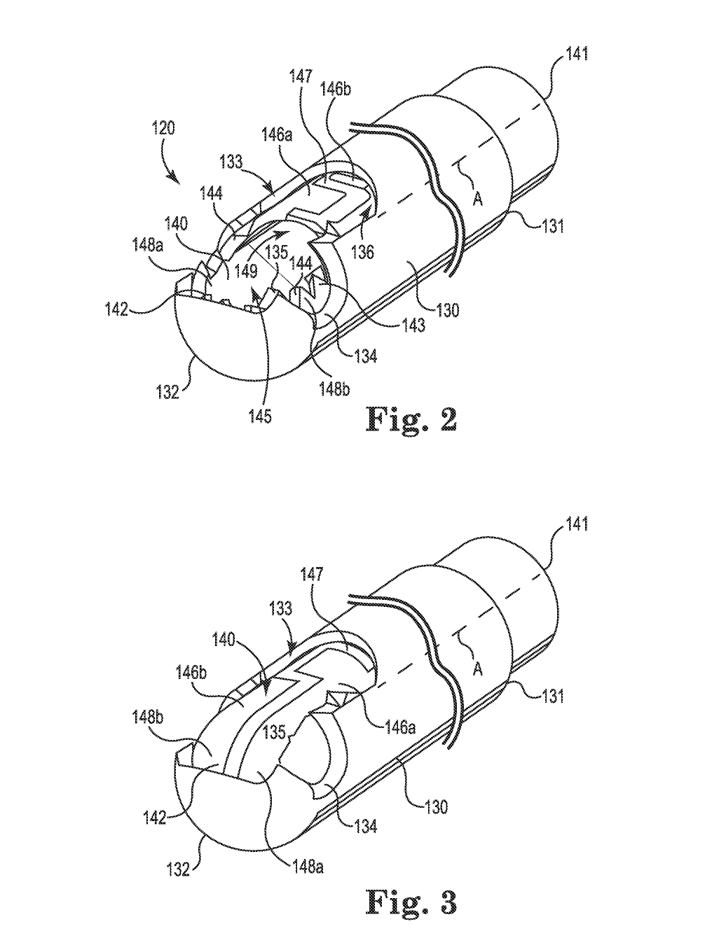

FIGS. 2 and 3 show perspective views of outer shaft 130 and inner shaft 140 of device 100 with inner shaft 130 in a first position (FIG. 2) and a second position (FIG. 3) with respect to outer shaft 140. The outer shaft 130 extends from a proximal end 131 to a distal end 132 that includes a window or opening 133. Window 133 is defined by an outer shaft cutting edge or cutter 134, which comprises cutting teeth 135. The outer shaft 130 may be rigid or malleable or combinations thereof and may be made of a variety of metals and/or polymers or combinations thereof, for example stainless steel. The inner shaft 140 extends from a proximal end 141 to a distal end 142, with the distal end 142 exposed through the window or opening 133 of outer shaft 130. A lumen 136 of the outer shaft 130 is configured to carry fluid between an outer diameter of the inner shaft 140 and an inner diameter of the outer shaft 130. In FIG. 2, inner shaft 140 is depicted in a position such that an inner shaft cutting edge or cutter 143, comprising cutting teeth 144 is exposed to window 133. Cutter 143 further defines an inner shaft window or opening 145. Outer and inner shaft cutters 134 and 143 may move relative to one another in oscillation or rotation (or both) in order to mechanically cut tissue. For example, outer shaft cutter 134 may remain stationary relative to the hub 175 and button assembly 200 while the inner shaft cutter 143 may rotate about a longitudinal axis A of the device, thereby cutting tissue.

Inner shaft 140 is formed of two longitudinal portions or halves 146a and 146b, separated by an insulating layer 147. In one embodiment, as will be discussed in more detail below, assembly of portions 146a, 146b and insulating material 147 is performed using an overmold process. In general, insulating layer 147 electrically isolates portions 146a and 146b and can be formed of a suitable non-conductive polymer. Upon final assembly, each of the portions 146a and 146b form an electrode 148a and 148b, respectively, at the distal end 142 of shaft 140. Rotation of inner shaft 140 may be achieved via manipulation of hub 175 (FIG. 1) that can orient the inner shaft 140 relative to the outer shaft 130 and may additionally allow for locking of the inner shaft relative to the outer shaft in a desired position, i.e., inner shaft 140 may be locked in position when cutter 143 is facing down with an outer surface of distal end 142 facing up. As described above, hub 175 may be connected to a handle or handpiece 177 which may be controlled by an IPC 179. Alternatively, the hub 175 and/or handle portions may be manipulated manually. Inner shaft 140 may be selectively rotated to expose a larger surface area of electrodes 148a, 148b, through opening 133 of outer shaft 130, as shown in FIG. 3.

As depicted in FIG. 3, inner shaft 140 is positioned such that the inner shaft cutter 143 is facing the interior (not shown) of outer shaft 130 and may be said to be in a downward facing direction and comprise a downward position. In the downward position, tissue is shielded from the inner shaft cutter 143. During hemostasis (via energy delivery through electrodes 148a, 148b), energy is delivered to tissue without attendant risk that the cutting teeth 144 of the inner shaft 140 will diminish the efforts to achieve hemostasis. Device 100 may thus comprise two modes: a cutting or debridement mode and a sealing or hemostasis mode and the two modes may be mutually exclusive, i.e., hemostasis is achieved via energy delivery to tissue while cutters 134, 143 are not active or cutting. As described below, energy may be advantageously delivered simultaneously with a fluid such as saline to achieve an optimal tissue effect by delivering controlled thermal energy to tissue.

When the inner shaft 140 is oriented such that the cutter 143 is in the downward position, rotating inner shaft 140 approximately 180 degrees relative to the outer shaft 130 will expose inner shaft cutter 143 and inner shaft opening 145 through the outer shaft opening 134. When the inner shaft cutter 143 is positioned as shown in FIG. 2, the inner shaft cutter 143 may be said to be in an upward position. The inner shaft opening 145 is fluidly connected to an inner shaft lumen 149 that extends from the inner shaft opening 145 to the proximal end 141 of inner shaft 140 and may be fluidly connected with the suction source 172. With this configuration, tissue cut via inner and outer shaft cutters 143, 134 may be aspirated into the inner shaft lumen 149 through the inner shaft opening 145 upon application of suction source 172, thereby removing tissue from a target site.

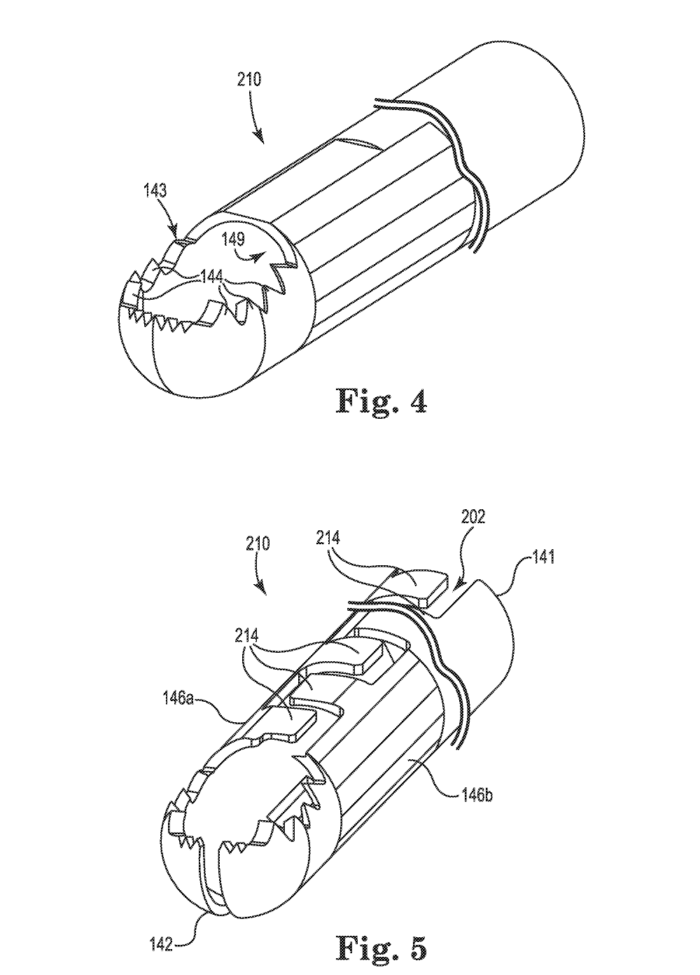

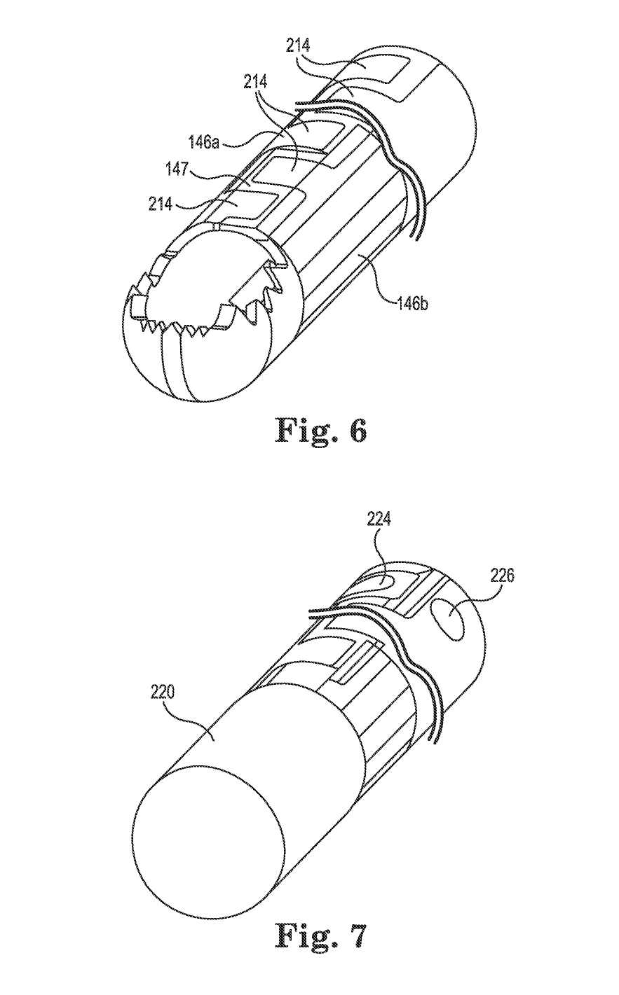

FIGS. 4-8 illustrate successive steps in forming outer shaft 140. In FIG. 4, a tube or tubular body 210 is formed that defines cutter 143 having teeth 144 and the window 145. Tube 210 can be made of a variety of conductive materials such as a metal alloy, for example stainless steel. Tube 210 is then cut into the two longitudinal portions 146a and 146b that extend from proximal end 141 to distal end 142 as illustrated in FIG. 5. In particular, the tube 210 is cut to form a slot 212, which can be of various configurations. In the illustrated embodiment, slot 212 forms opposed fingers 214 along a length of the tube 210. Fingers 214 interlock with one another to enhance structural integrity upon assembly of the shaft 140. As shown in FIG. 6, portions 146a and 146b are then positioned in a mold wherein insulating material is positioned in the mold to form insulating layer 147, which secures portions 146a and 146b together. In particular, at least a portion of the insulating material is positioned between the portions 146a and 146b. In FIG. 7, a mask 220 is positioned over a distal end 222 of the tube 210 and corresponding contact point masks 224 and 226 are positioned over a proximal end 228 of tube 210. With masks 220, 224 and 226 in place, an insulating coating is applied to the tube 210. The insulating coating can be formed of, for example, fluorinated ethylene propylene (FEP), polytetrafluoroethylene (PTFE), parylene or any other material suitable as a non-conductive or electrically insulative material.

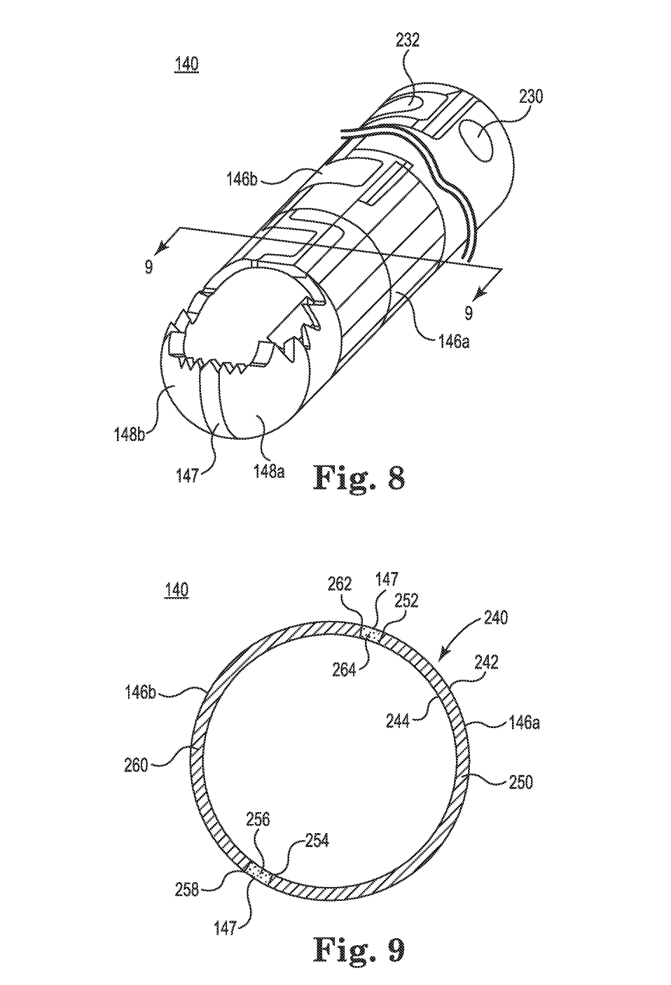

After coating, then, as illustrated in FIGS. 8 and 9, tube 210 has been formed into shaft 140 and is covered with an insulating layer, wherein electrodes 148a, 148b and contact points 230, 232 are exposed. Contact points 230 and 232 are electrically coupled with electrodes 148a and 148b, respectively. In addition, the contact points 230 and 232 are selectively electrically coupled with power source 162 through button activation cell 202 in order to deliver RF energy from source 162 to electrodes 148a and 148b. As illustrated in FIG. 9, which is a sectional view perpendicular to the longitudinal axis A of rotation for inner shaft 140, inner shaft 140 forms a tubular body 240 along its length that defines an outer diameter 242 and an inner diameter 244. A thickness of the tubular body 240 is defined between the outer diameter 242 and inner diameter 244. The thickness of tubular body 240 is constant throughout a circumference of the body 240, although other structures can be utilized. A structure of the thickness can be defined as including a first, electrically conductive arcuate section 250 formed of portion 146a, extending from a first joint 252 to a second joint 254 in the tubular body 240. A second, non-conductive arcuate section 256 formed of insulating material 147, extends from the second joint 254 to a third joint 258. A third, electrically conductive arcuate section 260 formed of portion 146b extends from the third joint 258 to a fourth joint 262. A fourth, non-conductive arcuate section 264 extends from the fourth joint 262 to the first joint 252. Joints 252, 254, 258, 262 of the tubular body 240 are illustrated as abutting in a linear fashion with respect to adjacent sections. In other embodiments, other configurations for the joints can be utilized, for example by utilizing lips, notches, fingers and the like.

Electrodes or 148a and 148b comprise bipolar electrodes and may comprise wet or dry electrodes. Electrodes 148a and 148b may be used to deliver any suitable energy for purposes of coagulation, hemostasis or sealing of tissue. Electrodes 148a and 148b are particularly useful with fluid such as saline provided by fluid source 152 (FIG. 1) which may be emitted near the outer shaft opening 133. Outer shaft opening 133 is fluidly connected to the outer shaft lumen 136. Lumen 136 extends from outer shaft opening 133 to the proximal end region 110 of device 100 and may be fluidly connected to the fluid source 152 (FIG. 1). Thus, fluid can be delivered to the opening 133 of outer shaft 130 and interacts with electrodes 148a, 148b. In this manner, electrodes 148a and 148b can advantageously provide Transcollation® sealing of tissue when used with the Transcollation® sealing energy supplied by the Aquamantys System, available from the Advanced Energy Division of Medtronic, Inc. With respect to “wet” RF coagulation technology, a variety of different technologies can be utilized including technology for sealing tissue described in U.S. Pat. Nos. 6,558,385; 6,702,810, 6,953,461; 7,115,139, 7,311,708; 7,537,595; 7,645,277; 7,811,282; 7,998,140; 8,048,070; 8,083,736; 8,216,233; 8,348,946; 8,361,068; and 8,475,455 (the entire contents of each of which is incorporated by reference). These patents describe bipolar coagulation systems believed suitable for use in the present invention. Other systems for providing a source of energy are also contemplated.

Returning to FIG. 1, when fluid from fluid source 152 is provided through lumen 136 of the outer shaft 130, the fluid may travel between the outside diameter of the inner shaft 140 and the inside diameter of the outer shaft 130 to the distal end 120 of device 100. Fluid travels distally down the lumen 136 of outer shaft 130 and may “pool” in an area as defined by the opening 133 of outer shaft 130. Pooling of fluid at the electrodes 148a, 148b allows for effective interaction between the fluid and the electrodes which in turn can provide effective and advantageous sealing of tissue, and in particular may provide effective Transcollation® sealing of tissue. In an alternative embodiment, an external tube mounted to an outer surface of outer shaft 130 for delivery of fluid can be utilized.

With continued reference to FIG. 1, electrodes 148a and 148b are situated in an area generally centrally located with respect to the outer shaft opening 133 when inner shaft cutter 143 is in a downward position. This generally central location of the electrodes 148a, 148b allows for energy delivery to adjacent tissue. In other words, after inner shaft cutter 143 and outer shaft cutter 134 are rotated or oscillated relative to one another to cut tissue, rotating inner shaft cutter 143 to the downward position to expose electrodes 148a, 148b and deliver energy through the electrodes 148a, 148b can allow for hemostasis in an area generally central to where debridement or cutting of tissue had taken place. The generally centered electrodes 148a, 148b allow for energy to essentially travel or radiate outwardly from the electrodes 148a, 148b to coagulate the approximately the entire area of tissue previously cut. In other words, energy, and particularly RF energy may be provided at the center or near center of a portion of tissue previously cut or debrided.

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1. A device for use with an energy source, comprising:
an outer tubular shaft defining a lumen and a window;
an inner tubular shaft extending from a proximal end to a distal end, comprising:
a first longitudinal portion extending from the proximal end to the distal end and formed of a conductive material;
a second longitudinal portion extending from the proximal end to the distal end and formed of a conductive material; and
an insulating layer extending from the proximal end to the distal end and disposed between the first longitudinal portion and the second longitudinal portion, wherein the proximal end is configured to be electrically connected to the energy source and the distal end defines a first electrode and a second electrode electrically coupled with the first longitudinal portion and the second longitudinal portion, respectively, the first electrode and the second electrode exposed at the window.

2. The device of claim 1, wherein the distal end forms a cutter including a plurality of teeth.

3. The device of claim 1, wherein, when viewing a cross section of the inner shaft, a thickness of the shaft includes two electrically conductive arcuate sections formed by the two portions and two non-conductive arcuate sections formed by the insulating material.

4. The device of claim 3, wherein the thickness is constant about a circumference of the inner shaft.

5. The device of claim 1, wherein each of the first and second longitudinal portions include a plurality of fingers interlocking with one another along a length of the tubular shaft.

6. The device of claim 1, wherein an insulating coating is positioned on an outer diameter of the tubular shaft.

7. A debridement device comprising:
an outer shaft defining a lumen and an outer shaft cutter defining a window in the outer shaft;
an inner shaft rotatably disposed within the lumen of the outer shaft, the inner shaft including two portions extending longitudinally from a proximal end to a distal end of the inner shaft, an insulating layer disposed between the two portions such that the two portions are electrically insulated from one another, and an inner shaft cutter selectively exposed at the window.

8. The debridement device of claim 7, wherein the distal end of the inner shaft defines two electrodes electrically coupled with the two portions, respectively.

9. The debridement device of claim 7, wherein, when viewing a cross section of the inner shaft, a thickness of the shaft includes two electrically conductive arcuate sections formed by the two portions and two non-conductive arcuate sections formed by the insulating material.

10. The debridement device of claim 9, wherein the thickness is constant about a circumference of the inner shaft.

11. The debridement device of claim 7, wherein the inner shaft cutter includes a plurality of teeth.

12. The debridement device of claim 11, wherein the inner shaft and outer shaft cutters are configured to move relative to one another to mechanically cut tissue in a cutting mode.

13. The debridement device of claim 7, wherein each of the two portions include a plurality of fingers interlocking with one another along a length of the inner shaft.

14. The debridement device of claim 7, wherein an insulating coating is positioned on an outer diameter of the inner shaft.

15. The debridement device of claim 7, wherein each of the two portions comprises contact points on the proximal end of the inner shaft that are selectively coupleable to an energy source.

16. The debridement device of claim 15, wherein the energy source comprises bipolar RF energy.

17. The debridement device of claim 7, further comprising an outer shaft lumen between an inner diameter of the outer shaft and an outer diameter of the inner shaft that is configured to allow fluid flow between the inner shaft and the outer shaft.

18. The debridement device of claim 7, further comprising a button activation assembly comprising an electrical contact for providing electrical communication of the two portions with a source of energy.

19. A surgical debridement system comprising:
a debridement device including:
a proximal end region and a distal end region;
an outer shaft defining a lumen and an outer shaft cutter defining a window in the outer shaft;
an inner shaft rotatably disposed within the lumen of the outer shaft, the inner shaft including two portions extending longitudinally from a proximal end to a distal end of the inner shaft, an insulating layer disposed between the two portions such that the two portions are electrically insulated from one another, and an inner shaft cutter selectively exposed at the window;
a source of power coupled to the proximal end region for driving the inner shaft relative to the outer shaft;
an energy source electrically connected to the bipolar electrode assembly; and
a fluid source fluidly connected to the distal end region.

20. The surgical debridement system of claim 19, further comprising a suction source fluidly connected to the distal end region.
